(19) United States
(12) Patent Application Publication
(10) Pub. No.: US 2002/0025319 A1
(43) Pub. Date: Feb. 28, 2002

(54) Antibodies that bind phosphatidyl serine and a method of their use

(76) Inventor: Peter Brams, San Diego, CA (US)

Correspondence Address: PILLSBURY WINTHROP LLP, 1600 TYSONS BOULEVARD, MCLEAN, VA, 22102, US

(21) Appl. No.: 09/805,217

(22) Filed: Mar. 14, 2001

Related U.S. Application Data
(60) Provisional application No. 60/189,050, filed on Mar. 14, 2000.

(51) Int. Cl. A61K 39/395; G01N 33/574; C07K 16/46; C07K 16/44

(52) U.S. Cl. 424/178.1; 424/142.1; 435/7.23; 530/388.1

(57) ABSTRACT

The present invention relates to novel monoclonal antibodies that specifically bind to the phospholipid phosphatidyl serine and methods for treating a neoplastic disorder. Further, the phosphatidyl serine specific monoclonal antibodies of the invention can be used to determine the presence of tumors and/or cancer cells and for molecular targeting of chemotherapeutic drugs, toxins or radionuclides to tumors and/or cancer cells. Also, kits for detecting phosphatidyl serine specific tumors or cancer cells are provided.

### CROSS-REFERENCE TO RELATED APPLICATION

This application claims priority from U.S. Ser. No. 60/189,050, filed on Mar. 14, 2001 and is incorporated by reference herein.

### FIELD OF THE INVENTION

The present invention relates to novel monoclonal antibodies that specifically bind to the phospholipid phosphatidyl serine. These monoclonal antibodies can be used to determine the presence of tumors and/or cancer cells and for molecular targeting of chemotherapeutic drugs, toxins or radionuclides to tumors and/or cancer cells. More particularly, the present invention relates to a method for promoting a therapeutic immune response against tumors and/or cancers using these anti-phosphatidyl serine antibodies. The present invention also relates to kits for detecting phosphatidyl serine specific tumors or cancer cells.

### BACKGROUND OF THE INVENTION

The immune system can be harnessed to enhance its anti-tumor activities for the eradication of tumor cells and the prevention of metastasis by (i) targeting tumor associated antigens (TAA) that might be specifically recognized by antibodies or reactive T cells, and by (ii) blocking the tumors immunosuppressive activity by neutralizing immunosuppressive soluble factors or by enhancing co-stimulation.

In response to antigens such as TAAs, the immune system is capable of producing two types of antigen-specific responses, cellular and humoral responses. Clinically, in cellular responses, T cell epitopes are targeted by cytotoxic T lymphocyte (CTL)-inducing vaccinations, and in humoral responses, B cell epitopes are targeted with passive or active antibody therapy (the latter by use of anti-idiotype antibodies). It has long been appreciated that the development of humoral immunity against most antigens requires not only antibody-producing B lymphocytes but also the involvement of helper T lymphocytes. (Mitchison, Eur. J. Immunol., 1:18-25 (1971); Claman and Chaperon, Transplant Rev., 1:92-119 (1969); Katz et al., Proc. Natl. Acad. Sci. USA, 70:2624-2629 (1973); Raff et al., Nature, 226:1257-1260 (1970)).

TAAs that can be targeted with antibodies generally fall into 3 categories: (1) Asymmetrically expressed antigens, which are expressed on one surface of the membrane or in one direction in normal cells, such as prostate specific antigen (PSA). Transformed cells “loose” the asymmetry. (2) Embryonic antigens; e.g., carcinoembryonic antigen (CEA) that are re-expressed in the transformed phenotype. (3) Tumor-associated viral antigens, such as EBV, HPV and HHV-III.

Ideally, treatment of cancer would be directed to a common “pan tumor specific antigen” of high penetrating distribution that does not induce the emergence of resistant phenotypes. While such a target has yet to be found, it appears that the “atypical” appearance of phosphatidyl serine (PS) in the cell's outer leaflet can be exploited towards this purpose.

Upon cell activation, apoptosis, and malignant transformation a redistribution of membrane phospholipids occurs that results in the appearance of PS at the cells outer leaflet. This phenomenon does not appear to be restricted to a particular cell type, as it is seen in virtually all cells undergoing apoptosis (Fadok et al., J.Immunol. 148:2207-2216 (1992); Martin et al., J.Exp.Med. 182:1545-1556 (1995); Koopman et al., Blood 84:1415-1420 (1994)) and in many different types of tumors (Utsugi et al., Cancer Res. 51:3062-3066 (1991); VanDeWater et al., Cancer Res. 45:5521-5525 (1985); Sugimura et al., Blood Coagul. Fibrin. 5:365-373 (1994); Rao et al., Throm. Res. 67:517-531 (1992)). With the exception of “activation-dependent” exposure of PS in platelets (Zwaal et al., Blood 89:1121-1132 (1997); Zwaal et al., Mol. Cell Biochem. 91:23-31 (1989); Bevers et al., Biochim. Biophys. Acta 736:57-66 (1983)), certain endothelial cells (Qu et al., Biochem. J. 317:343-346 (1996)) and placental cells (Katsuragawa et al., Amer. J. Obst. Gynecol. 172:1592-1597 (1995); Rand et al., Amer. J. Obst. Gynecol. 171:1566-1572 (1994); Rand et al., Amer. J. Obst. Gynecol. 177:918-923 (1997)), PS exposure can be considered as a pan tumor specific antigen.

Because the expression of PS on cell surfaces is an indication that the cell is destined for phagocytic removal, PS exposure could be an important component of immune surveillance (Savill et al., Immunol. Today 14:131-136 (1993); Hannun et al., Blood 89:1845-1853 (1997); Fadok et al., Current Biology 8:R-R (1998)). Clearly, however, this activity is not effective in cancer patients. Although the reasons for this are not known, it could be due to the immunosuppressive effects of soluble lymphokines secreted by “successful” tumors or because the reticuloendothelial system cannot manage large tumor burdens. It would appear, therefore, that the induction of a specific immune response to PS could augment anti-tumor activity by inducing direct tumor cell killing or by enhancing macrophage recognition of tumor cells through Fc receptor-dependent phagocytosis.

PS is not a classical antigen. It is only ˜800 dalton, and because of its structural similarities to other phospholipids, antibodies must be directed to its phosphoserine moiety. Surprisingly, under certain conditions, PS can induce an immune response. Lipid antibodies are naturally found in patients with connective tissue diseases, particularly systemic lupus erythematosus (Asherson et al., J. Invest. Dermatol. 100:21S-27S (1993); Mackworth-Young et al., Immunol. Today 11:60-65 (1990)) and in anti-phospholipid syndrome (APS) (Matsuura et al., [letter] [see comments] Lancet 336:177-178 (1990); Roubey et al., Blood 84:2854-2867 (1994); Triplett et al., Throm. Res. 78:1-31 (1995)), a condition characterized by recurrent thrombosis. Although less frequent, anti-phospholipid antibodies (APA) have been detected in patients with malignancies, including leukemia, lymphoma, epithelial malignancies and thymoma (Becker et al., Cancer 73:1621-1624 (1994); Naldi et al., Dermatology 184:156 (1992)). Other studies have shown that APA levels were significantly higher in melanoma patients who received immunotherapy with interferon-α or bacillus Calmette-Guerin (Becker et al., Cancer 73:1621-1624 (1994); Herstoff et al., Archives of Dermatology 115:856-859 (1979)). Because auto-antibodies in patients with autoimmune diseases are capable of binding and killing cells that display the autoantigens, it is possible that the appearance of APA in some cancers, possibly as a consequence of the disease and/or treatment regimen, is responsible for the remissions commonly seen upon interferon-α treatment. Interestingly, there are reports suggesting that some patients with APS generate antibodies that bind and kill tumor cells in vitro (Fishman et al., Cancer 72:2365-2369 (1993); Fishman et al., Int. J. Oncology 10:901-904 (1997)).

Because of the autoimmune squealae characteristic of anti-phospholipid syndrome, one might anticipate that the generation of anti-PS responses would produce similar pathologies. However, the “autoimmune disease”-promoting antibodies in anti-phospholipid syndrome are directed against β2-glycoprotein 1 (β2GP-I), a plasma protein that binds anionic phospholipids. Thus, while β2GPI antibodies are pathogenic, lipid antibodies that do not bind glycoprotein I, do not seem to promote autoimmune pathologies. This suggests that appropriate selection of immortalized monoclonal antibodies would circumvent the detrimental activities associated with antibodies to β2GP1.

Since PS is not a classical protein antigen, it is not presented by MHC Class-I or MHC Class II. Traditionally, antibody responses to PS have been detected in autoimmune mice, and monoclonal antibodies have been generated through hybridoma formation from these mice. However, anti-PS responses can be induced by coupling the lipid hapten to a protein carrier in a manner analogous to other chemical haptens (e.g., dinitrophenyl and penicillin). Indeed, PS-specific responses using newly developed chemistries that preserve the integrity of the phospholipid's head group while coupling the moiety to a protein carrier have been generated (Diaz et al., Bioconjugate Chem. 9:250-254 (1998)). In addition, there are reports on both CD4+ and CD8+ T cells recognition of phospholipids (not PS) presented by CD1 molecules. These T cells might provide the help necessary to generate affinity matured IgG antibodies. This effect also raises the possibility that PS specific T cells could be induced through appropriate vaccinations (Hariharan et al., unpublished).

The alternative to targeting PS with antibody is to target the antigen by specific immunization. While this would primarily induce PS antibody responses, it could also induce specific CTL responses. Clinically, this approach could be more advantageous since both humoral and cellular responses could be recruited for a more comprehensive and sustained anti-tumor effect. One could also predict that PS coupled directly to a tumor-specific protein antigen could potentiate this response. While such an approach could result in broader tumor specificity and higher reactivities, there is an obvious possibility that blocking antibodies and anti-phospholipid syndrome-like autoimmune responses could be induced.

### SUMMARY OF THE INVENTION

While most therapeutic immune responses against cancer have been directed against unique tumor-specific antigens such as peptides or carbohydrates, it would be beneficial if effective anti-cancer therapies are developed to promote therapeutic anti-phosphatidyl serine responses that target the phospholipid at the surface of tumors or cancer cells.

Toward that end, the present inventors have discovered novel monoclonal antibodies that specifically bind to PS. Further, the present inventors have developed a method for promoting a therapeutic immune response against cancer using these anti-PS antibodies.

Thus, it is an object of the invention to provide novel monoclonal antibodies that specifically bind to phosphatidyl serine.

It is another object of the invention to provide novel monoclonal antibodies that specifically bind to phosphatidyl serine with an affinity ranging from 10⁻⁸M to 10⁻⁹M.

It is yet another object of the invention to provide a novel composition comprising, in a pharmaceutically acceptable carrier, monoclonal antibodies that specifically bind to phosphatidyl serine.

It is a more specific object of the invention to provide a novel method for promoting a therapeutic immune response against tumors or cancers comprising administering a therapeutic amount of a phosphatidyl serine specific monoclonal antibody of the invention to a host.

It is yet another object of the invention to provide a novel method for killing tumors or cancers comprising administering a therapeutic amount of a phosphatidyl serine specific monoclonal antibody of the invention to a host.

### BRIEF DESCRIPTION OF THE FIGURES

FIG. 1: Rabbits were immunized with PS-β2GP1 emulsified in PROVAX™ two weeks apart. One week after the second immunizations the rabbits were bled, and the activity to PS-OVA and to OVA was tested by ELISA.

FIG. 2: Two Cynomolgus monkeys were immunized with PS linked to KLH and to BSA. The monkeys were bled at the dates indicated and a serial dilution of the sera tested for activity to PS-OVA by ELISA. The immunogen injected at the time of bleeding is indicated in the box below the bleed dates.

FIG. 3: ⁵¹Cr labeled DHL4 cells were incubated with the antibodies listed, for 30 minutes at 37° C., before rabbit complement was added at a final concentration of 6.25%. Release of ⁵¹Cr into the supernatant was determined. The percent killing was calculated after subtracting spontaneous ⁵¹Cr release, relative to maximal release achieved by addition of 1% Triton X 100 final concentration. c2B8 is a humanized anti-human CD20 IgG1 antibody. 6D1 is a mouse γ2b, 1 anti-human CTLA4 antibody. Monkey IgG was purified from a commercially available serum. Monkey 1155 was immunized with PS.

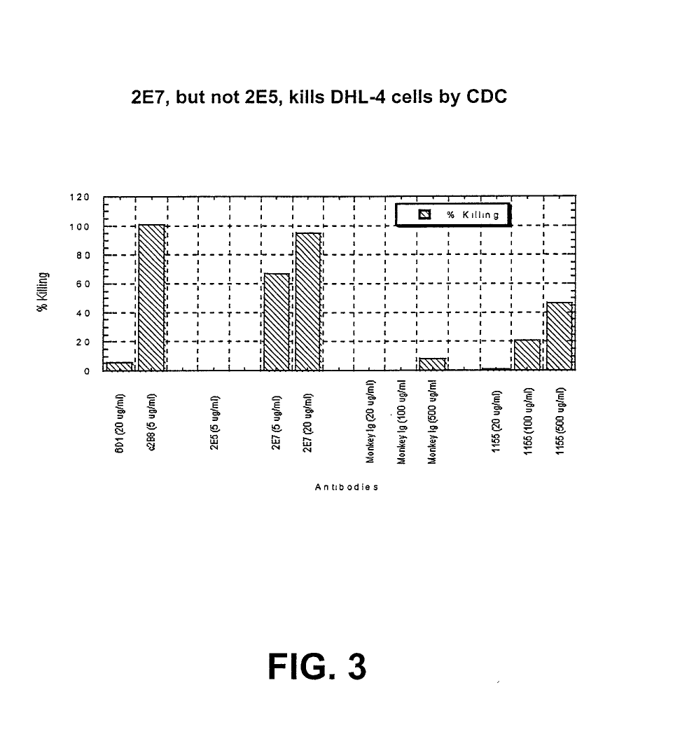

FIG. 4: Protection of SCID mice against SKW lymphoma induced lethality by polyclonal monkey anti-PS antibodies. Groups of SCID mice were inoculated with 3×10⁶ B lymphoma cells intravenously on day 0. On day 1, 7, 14 and 32, intra-peritoneal inoculation of purified polyclonal monkey anti-PS antibodies or IgG fraction obtained from pool of naive monkey serum (500 ug/mouse). On day 32, only the non-sick animals received the antibody inoculation. At indicated time points animals were monitored for hind-leg paralysis and death.

FIG. 5: Anti-tumor Activity of PS-BSA Against the L1210 Tumor System. Active immunization of mice with PS-BSA conjugate mixed in PROVAX™ in L1210 tumor system. Groups of C57BL/6 mice (6 per group) were inoculated with L1210 cells (1×10⁶). On days −20, −10 and +1, the mice were immunized with 100 μg of PS-BSA in PROVAX™ and tumor growth was measured every 3-4 days.

FIG. 6: Binding of purified anti-PS antibodies 2E5 and 2E7 to PS-OVA/OVA by ELISA.

FIG. 7: DHL-4 cells were incubated for 30 minutes at 0° C. with 2E7 (˜50 ug/mL), washed, and then stained with FITC-goat anti-mouse Ig. a) Cell stained with 2E7, b) Cells stained with FITC goat anti-mouse Ig only.

FIG. 8: Binding of 2E7 to PS-OVA in BIACore. At time 130 seconds 2E7 is added, at time 190 addition of 2E7 is stopped. Half-maximal binding appears at time 220.

### DETAILED DESCRIPTION OF THE INVENTION

Most attempts to promote a therapeutic immune response against cancer have been directed towards unique, tumor-specific, peptide or carbohydrate antigens. Little or no attention, however, has been given to the possibility that specific anti-lipid responses might also be exploited for this purpose. Although phospholipids are ubiquitous, it is clear that the organization and membrane sidedness of individual lipid species is not random but is controlled by transport mechanisms that maintain specific transmembrane lipid distributions (Pagano et al., Current Opinion in Cell Biology 2:652-663 (1990); Tang et al., Science 272:1495-1497 (1996)). Recent data suggests that while membrane organization is tightly regulated over the life span of the cell, normal lipid distributions are not maintained upon the cell's acquisition of several pathologic phenotypes (Zwaal et al., Blood 89:1121-1132 (1997)). This is particularly evident for senescent (Connor et al., J. Biol. Chem. 269:2399-2404 (1994); Boas et al., Proc. Natl. Acad. Sci. (USA) 95:3077-3081 (1998); Geldwerth et al., J. Clin. Invest. 92:308-314 (1993)), apoptotic (Fadok et al., J. Immunol. 148:2207-2216 (1992); Martin et al., J. Exp. Med. 182:1545-1556 (1995); Koopman et al., Blood 84:1415-1420 (1994)) and tumorigenic cells (Utsugi et al., Cancer Res. 51:3062-3066 (1991);VanDeWater et al., Cancer Res. 45:5521-5525 (1985); Sugimura et al., Blood Coagul. Fibrin. 5:365-373 (1994); Rao et al., Throm. Res. 67:517-531 (1992)), where a fraction of the phosphatidylserine (PS) redistributes from its normal location in the cell's inner leaflet to the cell's outer leaflet . This condition raises the possibility that PS present in the cell's outer leaflet can serve as a target for therapeutic intervention. The studies outlined below focus on the generation of therapeutic anti-PS responses that target the phospholipid at the cell surface of cancer cells.

Thus, in one respect the invention involves the discovery of PS-specific monoclonal antibodies and the use of such antibodies to effectively treat or eliminate tumors and/or cancers. These tumors and/or cancers include, but are not limited to those associated with lymphomas, leukemias, carcinomas, adenocarcinomas, sarcomas and myelomas. In particularly preferred embodiments, the invention may be used to treat patients suffering from B cell non-Hodgkin Lymphoma. Other neoplasms that preferably may be treated with the compounds and compositions of the instant invention comprise B cell chronic lymphocytic leukemias (CLL) and non T cell accute lymphoblastic leukemias (ALL). Those skilled in the art will readily be able to identify other neoplastic disorders that are susceptible to therapeutic intervention using the disclosed techniques and compounds based on the diagnostic protocols provided herein.

Preferably, such antibodies will specifically bind to PS with an affinity of at least 10⁻⁸M, more preferably from 5×10⁻⁹ to 10⁻⁹M, and most preferably from 5×10⁻¹⁰M to 10⁻¹⁰M.

In accordance with one embodiment of the invention, there is provided a method for promoting an immune response against tumors or cancers comprising administering a therapeutic amount of a phosphatidyl serine specific monoclonal antibody of the invention.

Essentially, a therapeutic amount of the anti-PS antibody of the invention is administered to a patient with a PS positive tumor or cancer. The dose of the anti-PS antibody to be administered can be determined by methods well known in the art. By binding to PS on the surface of tumor or cancer cells, the antibody will promote specific immune responses. For instance, some tumor cells are killed in vitro by a process involving antibody coating or opsonization which induces either phagocytosis by macrophages or antibody-dependent cell-mediated cytotoxicity (ADCC) in the presence of macrophages, natural killer cells or neutrophils.

Monoclonal antibodies can also show therapeutic activity against specific cells, e.g., malignant tissues based on the interaction of the Fc portion of the antibody heavy chain with other components of the immune system, such as the complement cascade or by binding to Fcγ receptors or various cytotoxic effector cell types.

Preferred antibodies to promote immune responses against PS-positive tumors or cancers include, but are not limited to, monoclonal antibodies, a mixture of monoclonal antibodies, polyclonal antibodies, a mixture of polyclonal antibodies, or a mixture of monoclonal and polyclonal antibodies. Additional preferred antibodies include anti-PS antibodies produced, for example, in rabbits, mice, and rats.

Non-human monoclonal antibodies (e.g., murine monoclonal antibodies) typically lack human effector functionality, i.e., they are unable to, inter alia, mediate complement dependent lysis or lyse human target cells through antibody dependent cellular toxicity or Fc-receptor mediated phagocytosis. Furthermore, non-human monoclonal antibodies can be recognized by the human host as a foreign protein. Thus, in human patients, it is more preferably to use human anti-PS antibodies, humanized anti-PS antibodies, or an anti-PS antibody produced by any method known in the art can be used.

By “humanized antibody” it is meant an antibody which is less immunogenic in humans. This is achieved by various methods known in the art, for example, one can produce a chimeric humanized antibody by grafting the non-human variable domains which retain antigen binding properties onto a human constant region. Additional methods are disclosed in Morrison et al., Proc. Natl. Acad. Sci. 81: 6851-5 (1984); Morrison et al., Adv. Immunol. 44: 65-92 (1988); Verhoeyen et al., Science 239: 1534-1536 (1988); Padlan, Molec. Immun. 28: 489-498 (1991); and Padlan, Molec. Immun. 31: 169-217 (1994), all of which are hereby incorporated by reference in their entirety.

In other embodiments of the invention, unconjugated non-PS antibodies, antibodies conjugated to toxins, chemotherapeutic drugs or radionuclides are used in conjunction with the compounds, methods and compositions of the instant invention. For example, in the preferred embodiment the disclosed antibodies may be used in conjunction with Rituxan® (IDEC Pharmaceuticals, Sand Diego, Calif.) a chimeric anti-CD20 antibody the art binds to B cells. In yet other embodiments, radionuclides or toxins, such as Ricin A chain or Pseudomonas toxin, and chemotherapeutic drugs, such as adrianycin, can be conjugated to the anti-PS antibodies of the invention and administered to a mammal in need of treatment. Further, the anti-PS antibodies (either conjugated or unconjugated) may be used in conjunction with various anti-cancer drugs that may be administered simultaneously or before or after the antibodies of the instant invention. Some classes of drugs or agents that may be advantageously used in accordance with the instant invention include, but are not limited to: metabolic enzyme inhibitors (e.g., MTX, Tomudex) including Topisomerase enzyme inhibitors (podohylotoxins, e.g., etopside), anti-metabolites (e.g., fluorouracil) Porphyrin (gadolinium-texaphyzin) or DNA intercolators (e.g., Anthacyclins, Camptothecins, etc.). In other preferred embodiments the anti-PS antibodies (again in a conjugated or unconjugated state) may be used in combination treatment with chemotherapy (e.g. fuldarabin, etc.)or with the motherapy combination) (CHP or CHOP).

In addition, radiolabeled antibodies can be used. A variety of radionuclides such as iodine-131(¹³¹I), indium-131 (¹³¹In) or yttrium-90 (⁹⁰Y) can be conjugated to a monoclonal antibody of the invention. Alternatively, radiolabeled non-PS antibodies may be administered in combination with conjugated or non-conjugated PS antibodies. Those skilled in the art will appreciate one of the advantages to administering chemotherapeutic drug-conjugated antibodies or radiolabeled antibodies is “bystander” killing, i.e., neighboring tumor or cancer cells are also killed.

The anti-PS antibodies of the invention may be administered to a human or other animal in an amount sufficient to produce a therapeutic or prophylactic effect. Such antibodies of the invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables.

The route of administration of the antibody (or a fragment thereof) of the invention may be oral, parenteral, by inhalation or topical. The term parenteral as used herein includes intravenous, intraperitoneal, intramuscular, subcutaneous, rectal or vaginal administration. Subcutaneous and intramuscular forms of parenteral administration are generally preferred.

The daily parenteral and oral dosage regimens for employing compounds of the invention to prophylactically or therapeutically induce immunosuppression, or to therapeutically treat carcinogenic tumors will generally be in the range of about 0.05 to 500, but preferably about 0.5 to 100, milligrams per kilogram body weight per day and most preferably from about 1 to 20 milligrams per kilogram body weight per day.

The antibodies of the invention may also be administered by inhalation. By “inhalation” is meant intranasal and oral inhalation administration. Appropriate dosage forms for such administration, such as an aerosol formulation or a metered dose inhaler, may be prepared by conventional techniques. The preferred dosage amount of a compound of the invention to be employed is generally within the range of about 10 to 100 milligrams.

The antibodies of the invention may also be administered topically. By topical administration is meant non-systemic administration and includes the application of an antibody (or fragment thereof) compound of the invention externally to the epidermis, to the buccal cavity and instillation of such an antibody into the ear, eye and nose, and where it does not significantly enter the blood stream. By systemic administration is meant oral, intravenous, intraperitoneal and intramuscular administration. The amount of an antibody required for therapeutic or prophylactic effect will, of course, vary with the antibody chosen, the nature and severity of the condition being treated and the animal undergoing treatment, and is ultimately at the discretion of the physician. A suitable topical dose of an antibody of the invention will generally be within the range of about 1 to 100 milligrams per kilogram body weight.

As a tumor mass increases in size, there is a need for new blood vessels to “feed” the tumor. Endothelial cells, which are located in blood vessels, play an important role in angiogenesis or the growth of new blood vessels. Upon activation, the membranes of certain endothelial cells undergo a redistribution of membrane phospholipids resulting in the appearance of PS in the cell's outer leaflet.

Thus, in another embodiment of the invention therapeutically effective amounts of anti-PS antibodies can be administered to target and eliminate activated endothelial cells, thus, inhibiting angiogenesis and tumor growth.

In accordance with another embodiment of the invention, there is provided a method for detecting the presence of PS containing tumors or cancer cells. The method comprises incubating a sample suspected of containing phosphatidyl serine positive cancer or tumor cells with anti-PS antibodies and determining the presence of PS containing cells using techniques such as flow cytometry and cell ELISA.

PS exposure on the surface of the cells may be determined in a sample from any applicable source. Preferably, PS is determined in a blood sample. Other biological samples that may be used in conjunction with the instant invention include, but are not limited to, bone marrow, cerebrospinal fluid, cell culture, and tissue.

In a preferred embodiment of the present invention, PS is detected using a sandwich ELISA that utilizes anti-PS primary antibodies followed by secondary antibodies. For example, a mouse monoclonal antibody (Mab) (2E7) may be biotinylated or FITC labeled and used to detect PS exposed on the outer surface of cells. In other embodiments 2E7 bound to a surface is used to capture PS cells which are then detected using a labeled secondary antibody that interacts with a non PS cell surface antigen. The secondary antibody may also be a monoclonal antibody, a mixture of monoclonal antibodies, a mixture of polyclonal antibodies, or a mixture of monoclonal and polyclonal antibodies. These secondary antibodies are preferably coupled to a detectable label, using methods known in the art. Exemplary labels compatible with the present invention comprise radiolabels, flourescent labels and enzymes.

More particularly, one can use antibodies attached to any reporter, such as radiolabeled antibodies or antibodies directly conjugated to alkaline phosphatase (substrates include p-nitrophenyl phosphate (pNPP)), horseradish peroxidase (substrates include 5-aminosalicylic acid (5AS), 2-2′ azino-di-(3-ethylbenzthiazoline sulfonate), o-dianisidine, o-phenylenediamine dihydrochloride (OPD), and 3,3′5,5′-tetramethylbenzidne (TMB)), β-galactosidase (substrates include o-nitrophenyl-β-D-galactopyranoside (oNPG) and p-nitrophenyl-β-D-galactopyranoside (pNPG)), or luciferase.

Another embodiment of the invention involves a kit to detect the presence of PS, such as PS present in biological samples. Such a kit comprises an antibody directed against phosphatidyl serine and ancillary reagents for use in detecting the presence of PS positive tumors or cancers. Preferably, the kit contains any of: (1) a solid support, such as a microtiter plate, on which to bind a primary anti-PS antibody; (2) a solution containing the primary antibody; (3) buffer solutions to block unbound sites on the solid support and to wash the solid support; (4) a solution containing the labeled secondary antibody; and (5) PS− and PS+ cell control.

PS may be isolated and purified from samples by methods well known in the art such as affinity chromatography, immunoprecipitation, ammonium sulfate precipitation, ethanol precipitation, and anion or cation exchange chromatography. See Sambrook, et al., Molecular Cloning: A Laboratory Manual, 2^{nd} edition, Cold Spring Harbor Press, New York, 1989, which is incorporated herein by reference, for additional isolation/purification methods.

In another preferred embodiment, PS is isolated by immunoassays utilizing anti-PS antibodies which recognize PS. The antibodies may be polyclonal or monoclonal, preferably monoclonal. In a more preferred embodiment, the anti-PS antibodies are bound to a solid support. Materials that can be used as solid supports include, but are not limited to, polysaccharide based materials such as cellulose and dextran, silica, alumina, nylon, magnetic particles such as beads, and microtiter plates.

Having described the preferred embodiments of the present invention, one skilled in the art will recognize that modifications can be made to the preferred embodiments without altering the scope of the invention.

The following examples are provided to further describe the invention, however, the scope of the invention is not limited thereby.

### EXAMPLE I

### Establishment of PS as a Tumor Cell Marker

I(a). Expression and Detection of PS on Neoplastic Cell-lines in Vivo

To determine the expression of PS in vivo and demonstrate the concepts of the instant invention, ˜10⁶ DHL4, SKW and L1210 cell lines were injected subcutaneously in mice to establish solid tumors. When the tumors became palpable, they were excised and single cell suspensions were prepared. The cells then were analyzed for PS expression with Annexin V and selected anti-PS monoclonal antibodies.

All activated cells and tumor cells express PS to varying degrees. Analysis of PS expression of three lymphoma lines, the human lymphomas DHL4 and SKW, and the murine leukemia L1210, by flow cytometry for Annexin V-FITC binding (Koopman et al., Blood 84:1415-1420 (1994)), showed that <50% of the cells for any of the cells lines express PS above background. However, if PS expression on the outer membrane leaflet is an indication that the cell is undergoing apoptosis, then targeting such cells is not meaningful. Hence, we conducted studies to determine phenotypic distribution and characteristics between PS positive cells and PS negative cells in vitro. We separated the PS expressing DHL4 cells from PS non-expressing cells by cell sorting on a flow cytometer. Annexin V-FITC was used to identify the PS+ and PS− cells. Our data showed that the PS+ cells remained distinctly PS+ for up to two weeks, at what time they became phenotype indistinguishable from the parent population. The PS− cells also remained phenotypically distinct for up to two weeks when these cells likewise became indistinguishable from the parent population. Attempts to link PS-expression with cell cycle stage failed.

Our studies using tumor cell lines thus challenged the validity of the existing notion that not all PS+ cells will undergo apoptosis, and validated PS as a potential target in anti-cancer therapy.

### EXAMPLE II

### Generation and use of Polyclonal Antibodies to PS

II(a). Generation of Anti-PS Titers in Rabbits, Mice, and Monkeys

PS linked to a proteinous carrier, by means of the chemistry developed by Dr. Schroit et al. which conserve the functional head group (Diaz et al., Bioconjugate Chem. 9:250-254 (1998)), was used to immunize mice, rabbits and Cynomolgus monkeys. As proteinous carrier, we used KLH, BSA and β2GP1.

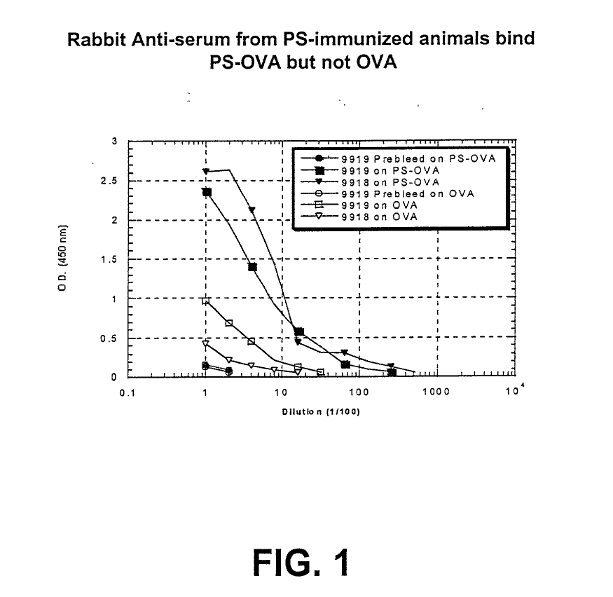

Rabbits were immunized with PS-BSA and PS-β2GP1 in PROVAX™ twice sub-cutaneously. The rabbits were bleed and serum prepared. The serum was tested for activity to PS linked to ovalbumin (PS-OVA) and to OVA by ELISA. The data show that antibodies specific for PS were induced in the animals (FIG. 1).

Balb/c mice immunized with with PS-β2GP1, showed strong PS-OVA vs. OVA activity, one reaching as high as 1:200,000 on PS-OVA vs. 1:200 on OVA (not shown). DBA mice were immunized with BSA-PS. The isotype of the anti-PS antibodies induced by the vaccination was primarily γ1, but γ2a was also enhanced significantly (Table I). These data indicate that natural anti-PS antibodies are present in mice.

TABLE I
Isotype of Anti-PS Responses After Immunizations Using PROVAX ™

| | | DBA mice | | Balb/c mice | |
|---|---|---|---|---|---|
| Isotype | Dilution | Control | 70.2 | Control | Test |
| IgG | 10 | 2.13 | 3.27 | 3.06 | 3.2 |
| | 40 | 1.08 | 2.26 | 2.13 | 2.2 |
| | 160 | 0.49 | 1.10 | 0.87 | 1.14 |
| | 640 | 0.30 | 0.43 | 0.36 | 0.46 |
| γ1 | 10 | 0.45 | 2.57 | 0.9 | 1.5 |
| | 40 | 0.23 | 1.32 | 0.34 | 0.65 |
| | 160 | 0.20 | 0.43 | 0.2 | 0.33 |
| | 640 | 0.18 | 0.24 | 0.18 | 0.2 |
| γ2a | 10 | 0.44 | 1.79 | 0.56 | 0.72 |
| | 40 | 0.16 | 0.57 | 0.24 | 0.27 |
| | 160 | 0.10 | 0.20 | 0.1 | 0.13 |
| | 640 | 0.09 | 0.16 | 0.1 | 0.1 |
| γ2b | 10 | 0.58 | 1.73 | 1.5 | 1.6 |
| | 40 | 0.22 | 0.44 | 0.5 | 0.47 |
| | 160 | 0.09 | 0.18 | 0.2 | 0.2 |
| | 640 | 0.07 | 0.11 | 0.1 | 0.1 |
| γ3 | 10 | 0.36 | 0.41 | 0.47 | 0.57 |
| | 40 | 0.16 | 0.17 | 0.2 | 0.22 |
| | 160 | 0.08 | 0.10 | 0.1 | 0.14 |
| | 640 | 0.10 | 0.09 | 0.1 | 0.1 |

Mouse sera diluted in buffer containing 10% fetal calf serum were plated out on an ELISA plate coated with PS-OVA and on a plate coated with OVA. Presence of antibodies of the various isotypes on the plates was revealed using rat antibodies specific for the 4 isotypes. Activity is listed in OD 450 on PS-OVA minus OVA.
DBA Control: Pooled immune-sera from mice injected with L1210 tumor, no immunizations.
70.2: Pooled immune sera from mice immunized w. PS-BSA twice, once before and once after injection of L1210 tumor cells.
Balb/c control: Pooled sera from pre-immune sera.
Test: Pooled sera from mice immunized twice with PS-bGP1.

To determine the feasibility of induction of anti-PS IgG responses in primates, to analyze the nature of such anti-PS antibodies, and in particular to study the safety of long term exposure to anti-PS antibodies, we immunized two Cynomolgus monkeys with PS-KLH followed by PS-BSA, both in PROVAX™. The main objective of this study was to determine the properties of the induced anti-PS antibodies in mediating killing of PS+ cells and to determine if any pathological antibodies were generated; i.e., if events similar to those observed in APS patients would be observed.

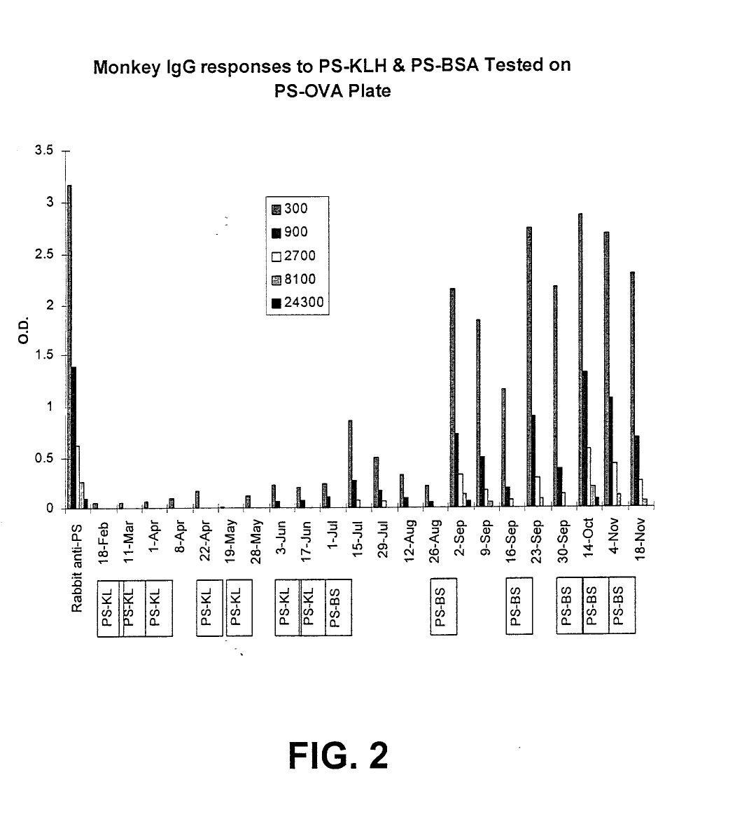

The anti-PS titers in the monkeys were barely detectable after immunization with PS-KLH, but increased to approximately 1:25,000 after three immunizations and increased further three fold after 5 more immunizations, when BSA was used as carrier for PS (FIG. 2). The isotype of the anti-PS antibodies were overwhelmingly γ1 (recruits effector functions), there were little anti-PS antibodies of other IgG subtypes. The anti-PS IgM titer was less than 1:250.

A series of appropriate blood parameters were studied on the bleeds. These parameters included hematocrit, white blood cell count, antibody coating of red blood cells (Coomb's test) and activated partial thromboblastin (APT) time or Russel Viper Venum test. There was no detectable reduction in neither white or red blood cell count, and except for one data point out of 22, Coomb's test was negative. The only tested parameter that was affected in the immunized monkeys was clotting time. One animal, 1155 showed a transient increase over normal in APT time, whereas animal 1157 showed an apparent sustained increase in APT time (Table II).

TABLE II
Activated Partial Thromboblastin (APT) Time

Date

| Animal # | 2/19 | 3/12 | 4/2 | 4/9 | 4/23 | 8/27 | 9/3 | 9/10 | 9/17 | 9/24 | 10/1 | 10/15 | 11/19 |
|---|---|---|---|---|---|---|---|---|---|---|---|---|---|
| PR1155 | 17.3 | 18.8 | 18.0 | 14.8 | 17.8 | 16.9 | 18.6 | 17.0 | 22.4 | 24.1 | 31.9 | 29.5 | 23.8 |
| PR1157 | 17.1 | 22.8 | 19.5 | 18.8 | 18.2 | 19.0 | 24.7 | 19.8 | 28.0 | 34.5 | 47.2 | 51.8 | N/A* |

The APT time, in seconds, was recorded for all bleeds taken during the study. APT times over 20 are highlighted in bold. Bleeds between 4/23 and 8/27 all had APTT below 20 seconds, and are not shown.
*N/A = not applicable, this monkey was sacrificed 11-04.

Cynomolgus monkey 1157 was killed in order to look for thrombi or hemorrhages by histology. Macroscopic histological analysis of the vascularature in the abdomen, kidney, lung, heart, meninges, brain, muscularature in legs and arms showed that all were free of any recent or old infarcts. The animal was evaluated to be in a grossly very good condition. Microscopic histology was only performed on the liver. The finding was that all sections were comparable to a liver from a normal animal.

II(b). Functional Activity of Anti-PS IgG Antibodies in Vitro and in Vivo

In order to determine if the antibodies generated in PS-immunized animals could confer anti-tumor protection, and whether targeting PS with antibodies or monoclonal antibodies would be effective, antibodies purified from the PS-immunized monkeys were compared to antibodies purified from non-immune animals. These antibodies were tested for their ability to mediate killing of PS+ cells via CDC and for their activity in an in vivo protection assay.

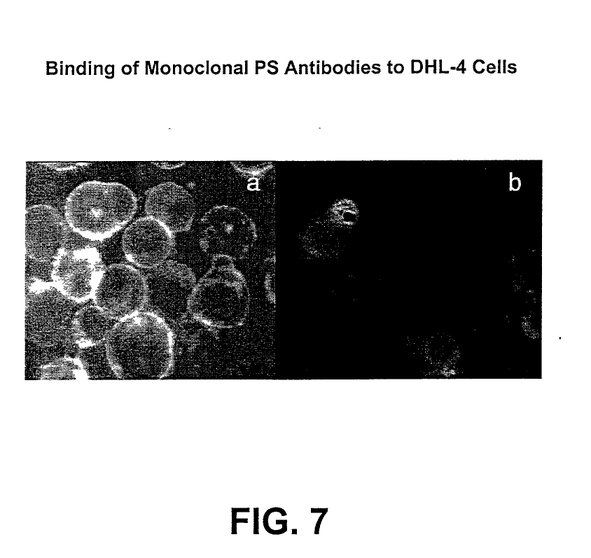

To test the ability of anti-PS antibodies to mediate complement dependent cell killing, we used a human lymphoma line, DHL4, as target. The cells were labeled with 51 chromium and incubated with the purified antibody preparations in the presence of complement. Antibodies purified from PS-immunized animals mediated killing by CDC, up to 70%, whereas antibodies purified from non-immune animals, mediated little to no killing, less than 10%, at 500 μg/ml (FIG. 7).

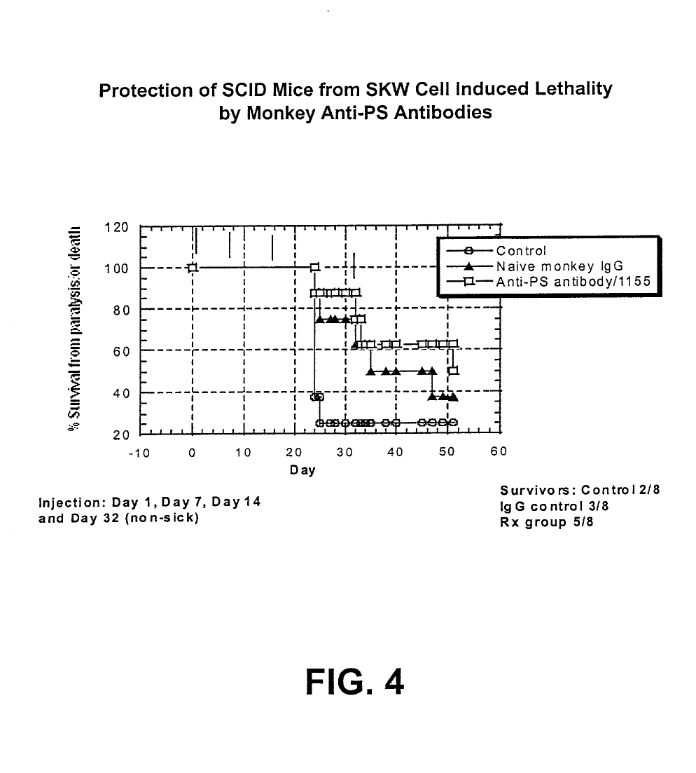

In a passive tumor protection study, SCID mice were inoculated with the human lymphoma cell line SKW and infused with purified monkey antibodies. Both the purified naive monkey IgG and the PS specific IgG antibody from the PS immunized monkey, delayed paralysis or death of the mice (FIG. 4). The anti-PS IgG preparation did perform superior to naive IgG, but the study indicates that the anti-PS antibodies naturally present in serum have protective activity. As the naive anti-PS antibodies mediate little CDC, this indicates that these antibodies might work by mediating phagocytosis.

The data support the notion that targeting PS and specifically targeting PS with antibodies could be of therapeutic significance.

### EXAMPLE III

### Production and Identification of Anti-PS Monoclonal Antibodies

III(a). Generate High Anti-PS Titers in PS-BSA/PROVAX™ Immunized Mice

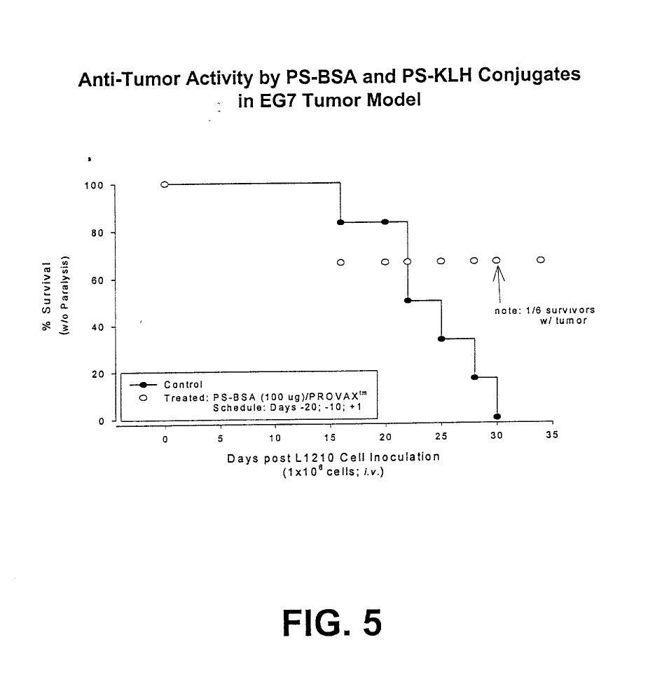

As discussed in Example II(a) mice were injected with PS-BSA to provide antibodies. The murine leukemia cell line L1210 was injected into the tail veins of tranquilized mice at a concentration of 1×10⁶ per mouse. The mice were immunized, as described in FIG. 5, with PS-BSA in PROVAX™ up to six times, two weeks apart. Each mouse was immunized with from 25 to 100 μg of PS-BSA in no more than 100 μl PROVAX™. Immunizations were done SC or IP.

Sedated mice were bled by orbital sinus rupture and less than 150 μl of blood were taken. Mice with tumors were sacrificed at the time they showed visible signs of paralysis. The mice that were “cured” were the mice that survive beyond day 45. A week later, the mice were bled by sinus orbital puncture and anti-PS serum titers were determined by ELISA as shown in Table I. “Cured” mice were analyzed for IgG titers to PS, and the strongest responders that were tumor-free were sacrificed for splenectomy, whereas the remaining mice were sacrificed at the completion of the study.

One of three mice that survived, was further immunized with PS-BSA emulsified in PROVAX™ by IP injections. The end dilution of anti-PS IgG titers from mouse 70.2.3, one of the mice pooled to form the titer labeled 70.2 in Table I, reached 2×10⁵ just prior to fusion.

III(b). Identify Anti-PS Antibodies through Binding to PS-OVA by ELISA

As discussed above, one primary means of identifying PS reactive antibodies is to assay for binding to PS linked ovalbumin (PS-OVA) but not to ovalbumin (OVA). Briefly, sera from the immunized mice was diluted in buffer containing 10% fetal calf serum and plated out on an ELISA plate coated with PS-OVA and on a plate coated with OVA. The presence of PS specific antibodies of the various isotypes on the plates was revealed using rat antibodies specific for the 4 isotypes in protocols well known in the art. See Table I (activity in Table I is listed in OD 450 on PS-OVA minus OVA).

III(c). Formation of Hybridomas from Tumor Survivors

The mice with the highest anti-PS titers were boosted with antigen in phosphate buffered saline (PBS). Three days later, the mice were sacrificed. The peritoneum of the mice was flushed with PBS and the media then sterilely aspirated out. The spleens were then taken out and pressed through fine wire mesh.

The resulting cell suspension was washed and mixed at a ratio of 5:1 with SP2/0 cells in exponential growth. Using standard protocols described in (Brams et al., J. Immunol. Methods, 98, 11-22 (1987)), lymphocytes from the mice with the highest anti-PS titers were subjected to hybridoma formation in PEG using 50% PEG 1500. After the fusion, the resulting cells were then split in two, half of which were plated in ˜800 96-well plate wells, while the other half was spun down by centrifugation and re-suspended into 50 ml methyl cellulose medium containing HAT (StemCell Technologies) and plated in 10 petri dishes. After 10-12 days, hybridoma clones were picked out of the petri dishes and transferred to medium containing HT.

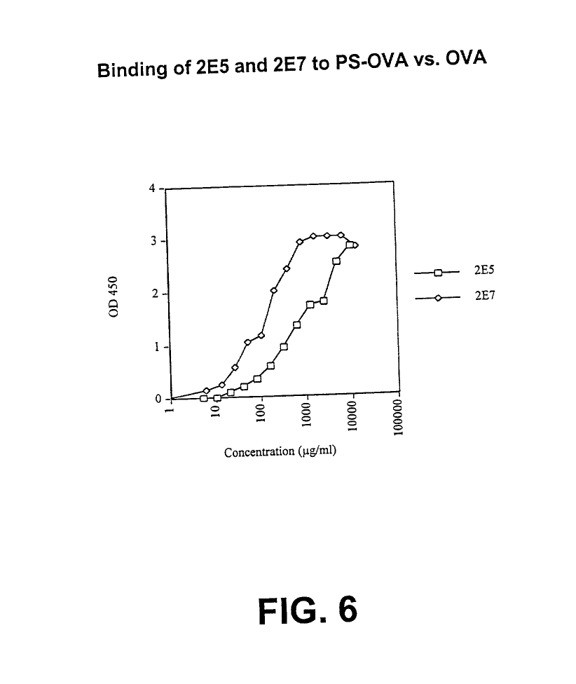

The spleen cells of mouse 70.2.3 was subjected to hybridoma formation using the fusion partner SP2/0 as described immediately above. Of more than 800 growing hybridomas, two produced antibody that bound PS-OVA but not OVA. The two clones were called 2E5 and 2E7 (FIG. 6), and the respective isotypes are γ,λ and γ2bκ. Half-maximal binding concentrations of 2E5 and 2E7 were determined to be approximately 650 ng/ml and 125 ng/ml, respectively.

Using a similar protocol, peritoneal CD5+-B cells were subjected to hybridoma formation and plated in 120 wells of 96 well plates.

III(d). Determine binding to PS positive vs. PS Negative Cells

Part of the identification and specificity criteria included binding of PS-antibodies to PS+ cells vs. PS− cells. Target cells; e.g., DHL4, were separated into PS+ and PS− populations by flowcytometry using Annexin V-FITC binding. The cells were then grown for three days to assure viability. Selected antibodies, including 2E5 and 2E7, were serially diluted, tested for binding to the two DHL4 cell populations by flowcytometry and compared to PS expression using Annexin V binding.

Several monoclonal PS antibodies, including both 2E5 and 2E7, bound PS-positive DHL-4 cells, but not PS negative cells (FIG. 7; PS-negative cells not shown). The antibodies bound with a similar distribution as Annexin V, but with less intensity. Based on antibody binding profiles, one antibody was selected for further development.

### EXAMPLE IV

### Characterization of PS-specific Monoclonal Antibodies

IV(a). Determine Affinity to PS Using Plasmon Resonance (BIACore)

One essential parameter in the initial characterization of an antibody is specificity and binding affinity, we included in the early stage selection process, binding of PS-OVA to solid phase PS-specific antibody in BIACore. A goat anti-human antibody preparation that binds approximately 5000 arbitrary units of human antibody was plated out on the solid phase chip. Supernatant containing PS-specific antibody was then added to the chip, and the chip was washed with PBS until equilibrium is established. Finally, PS-OVA was added and the on- and off- rates were determined. The antibodies with the most desirable binding characteristics were then selected for further analysis.

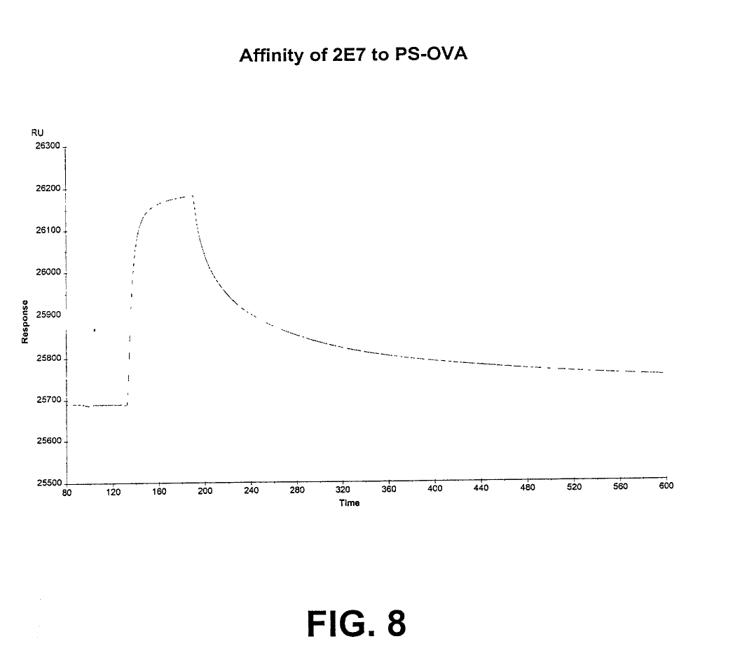

Among the antibodies having relatively desirable binding characteristics was monoclonal antibody 2E7. The data shows an unusually fast on rate followed by a fast off rate (FIG. 8). Affinity (avidity) was calculated to be between 10⁻⁸ and 10⁻⁹M.

IV(b). Complement Dependent Cytolysis Assays on PS+vs. PS-cells

In order to enable in vitro and in vivo functional tests on the selected antibody, the heavy chain isotype was determined using common immunochemical techniques employing commercially available kits. It will be appreciated that those antibodies of a γ2 isotype enable recruitment of effector functions and are particularly desirable in terms of the present invention. Accordingly, antibodies possessing such isotypes were selected for further testing and development.

Once the antibody was characterized as a γ2b antibody, it was tested for activity in complement dependent cytolysis (CDC) on PS+ vs. PS− cells of lymphoid origin. The γ2b antibody was tested on three cells lines, two human lymphomas, SKW and DHL4 and one murine leukemia cell line, L 1210, after each cell line was separated into PS+ and PS− populations. Briefly, cells were incubated at 5×10⁶ cells/ml in 200 μCi/ml ⁵¹Cr overnight. The cells were then washed free of excess ⁵¹Cr and incubated with a serial dilution of the anti-PS antibody in the range from 0.1 μg/ml to 20 μg/ml for 30 minutes at 37° C. in 96-well plates (10⁴/well). During the incubation period, rabbit complement (Cappell) at a final concentration of 6.25% was added. The release of ⁵¹Cr was determined after 90 minutes incubation. As a positive control for the human cells, we used c2B8. The murine γ2b anti-CTLA4 antibody was used as a negative control.

The experiment showed that 2E5, a γl antibody, did not effectively mediate killing by CDC, whereas 2E7, a γ2b antibody, did (FIG. 7). Based on the observation that 2E7 appeared to recruit effector functions it may be that this antibody would provide a particularly attractive candidate for commercial development.

IV(c). Macrophaze Phagocytosis Assay

In order to further characterize the antibody, it was also tested for the ability to mediate phagocytosis by macrophages on PS+ vs. PS− cells of lymphoid origin. The antibody was tested on three cells lines, two human lymphomas, SKW and DHL4 and on one murine leukemia cell line, L1210, after each cell line was separated into PS+ and PS− populations. The protocol is similar to the one described earlier (Balasubramanian et al., J. Biol. Chem., 272, 31113-7 (1997)), except that the target cells were labeled with 10 μM Vybrant dye (CFDA SE from Molecular Probes) according to the manufacture's protocol. Macrophages were isolated from a spleen cell preparation by cell adhesion to the bottom of the wells of the reaction plate with non adherent cells were removed by washing with buffer. For experiments targeting the human lymphomas, we used human spleen cell derived macrophages (e.g., spleen cell suspension acquired through the NIH sponsored Cooperative Human Tissue Network). For experiments targeting the murine leukemia, we used adherent cells derived from murine spleens. Briefly, spleen cells (6×10⁶/well of a 24-well plate) were plated out for 90 minutes. Non-adherent cells were removed by extensive washing with buffer. The Vybrant dye labeled cells were incubated with the adherent macrophages for 90 minutes in the presence of a serial dilution of the selected anti-PS antibody in a range from 0.1 μg/ml to 20 μg/ml. After incubation, non-adherent cells were washed off and the amount of dye transferred to the adherent macrophages was determined using a fluorometer from Molecular Devices. As positive control we used c2B8, an anti-CD20 antibody, with murine γ2b anti-CTLA4 antibody, 6D1, used as a negative control.

The results indicated that PS+cells underwent phagacytosis at a greater rate than PS− cells. This is consistent with the finding that PS+ is most often exposed in cells undergoing apoptosis and needing elimination. In view of such finding, the binding of anti-PS antibodies to PS+neoplastic cells could lead to more efficient eradication of the tumor by invoking complementary mechanisms such as CDC and Fe mediated cytolytic activity

IV(d). Half-life in Mice

In preparation for studies on the activity of 2E7 in human tumor protection models in SCID mice, 200 μg 2E7 was administered to 6 SCID mice each. The mice were bleed at various time points and the β half-life of 2E7 calculated. The half-life was calculated to be approximately 6 days. This value indicates that 2E7 is slowly but actively being depleted by the non-transformed murine environment, as the half-life of non-specific murine IgG is between 20 and 30 days.

IV(e). Amino Acid Sequence of 2E7

The sequence of the genes coding for 2E7 revealed that both the heavy and the light chain V regions have been published previously in separate papers. The light chain was part of a molecule that recognized e-aminocaprioic acid (Elliot et al., J.Immunol., 133, 2757-2761 (1984)), whereas the heavy chain was part of an antibody that recognized p-azaphenylarsanate (Efter et al., Ann. Inst. Pasteur Immunol., 1350, 17-30 (1984)). This remarkable degree of conservation indicates that 2E7 is produced by a CD5+ B cell. This could not be corroborated by staining with anti-CD5 as the fusion partner is highly CD5 positive. This notion has consequences for our future attempts to immortalize a better anti-PS antibody. CD5+ B cells are primarily found in the peritoneum and we will, therefore, in the future focus on IP immunizations and include generating hybridomas with cells from peritoneal washes as well as from spleen derived cells.

### EXAMPLE V

### Targeting Tumors with 2E7 in a Human Metastatic Model in SCID Mice

V(a). Tumor Protection Studies

In order to establish feasibility of targeting PS expressing tumors with passive immunotherapy, we tested the existing anti-PS antibody, 2E7, for the ability to protect SCID mice from human lymphoma-cell induced lethality. The model we used was the same as that used with monkey polyclonal antibodies as described in Example 11(b), a metastatic model in SCID mice, SKW.

A total of 8 SCID mice were used in each arm of the experiment. One million cells of a lymphoma cell line were injected IV through the tail vein on day 0. On days 1, 3, 5, 7, 9 and 11, the 2E7 antibody was injected IP in PBS at a concentration of 200 μg antibody per mouse per injection. The mice were monitored daily for hind leg paralysis. When paralysis was observed the mouse was sacrificed. As a positive control, we used c2B8, and as a negative control, we used 6D1. The negative control mice were killed before day 25, while c2B8 regularly delays onset of paralysis for more than 30 days.

Like c2B8, 2E7 was found to delay the onset of paralysis brought about by the injection of the lymphoma cells. This indicates that anti-PS antibodies may be used to eliminate PS+ neoplastic cells in vivo.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments which have been described in detail herein. Rather reference should be made to the appended claims as indicative of the scope and content of the invention.

What is claimed is:

1. An anti-phosphatidyl serine antibody or fragment thereof that binds the phospholipid phosphatidyl serine.

2. The anti-phosphatidyl serine antibody of claim 1, wherein said anti-phosphatidyl serine antibody is a monoclonal antibody.

3. The anti-phosphatidyl serine antibody of claim 2, wherein said monoclonal antibody binds the same epitope as 2E7 or competes with 2E7 for binding the phospholipid phosphatidyl serine.

4. The anti-phosphatidyl serine antibody of claim 2, wherein said monoclonal antibody is a chimeric, human or humanized antibody.

5. A composition for treating tumors, neoplasms or cancers comprising an anti-phosphatidyl serine antibody of claim 1 and a pharmaceutically acceptable carrier.

6. The composition of claim 5, wherein said antibody is a monoclonal antibody.

7. The composition of claim 6, wherein said monoclonal antibody binds the same epitope as 2E7 or competes with 2E7 for binding the phospholipid phosphatidyl serine.

8. The composition of claim 6, wherein said monoclonal antibody is a chimeric, human or humanized antibody.

9. A method of treating a neoplastic disorder comprising administering a therapeutically effective amount of an anti-phosphatidyl serine antibody or a fragment thereof to a mammal in need thereof.

10. The method of claim 9, wherein said anti-phosphatidyl serine antibody is a monoclonal antibody.

11. The anti-phosphatidyl serine antibody of claim 10, wherein said monoclonal antibody is a chimeric, human or humanized antibody.

12. The method of claim 10, wherein said anti-phosphatidyl serine antibody binds the same epitope as 2E7 or competes with 2E7 for binding the phospholipid phosphatidyl serine.

13. The method of claim 9, wherein said anti-phosphatidyl serine antibody is conjugated to a toxin, a drug or a radionuclide.

14. The method of claim 13, wherein said toxin is ricin A chain or Pseudomonas toxin.

15. The method of claim 13, wherein said anti-phosphatidyl serine antibody is conjugated with a chemotherapeutic drug.

16. The method of claim 13, wherein said anti-phosphatidyl serine antibody is conjugated with a radionuclide.

17. A method for inhibiting angiogenesis comprising administering a therapeutically effective amount of an anti-phosphatidyl serine antibody or a fragment thereof to a mammal in need thereof.

18. The method of claim 17, wherein said anti-phosphatidyl serine antibody is a monoclonal antibody.

19. The method of claim 18, wherein said monoclonal antibody is a chimeric, human or humanized antibody.

20. The method of claim 17, wherein said anti-phosphatidyl serine antibody binds the same epitope as 2E7 or competes with 2E7 for binding the phospholipid phosphatidyl serine.

21. The method of claim 17, wherein said anti-phosphatidyl serine antibody is conjugated to a toxin, a drug or a radionuclide.

22. The method of claim 21, wherein said toxin is selected from the group consisting of ricin A chain and Pseudomonas toxin.

23. The method of claim 21, wherein said anti-phosphatidyl serine antibody is conjugated with a chemotherapeutic drug.

24. The method of claim 21, wherein said anti-phosphatidyl serine antibody is conjugated with a radionuclide.

25. A method of detecting PS positive tumors or cancers comprising:
(i) incubating a sample with an antibody directed against phosphatidyl serine under conditions which allow the formation of an antigen-antibody complex; and
(ii) detecting said antigen-antibody complex.

26. The method of claim 25, wherein said sample is selected from the group consisting of blood, bone marrow and combination thereof.

27. A kit for analyzing a sample for the presence of PS positive cells comprising an antibody directed against phosphatidyl serine and ancillary reagents for use in detecting the presence of phosphatidyl serine.

28. The kit according to claim 27, wherein said kit further comprises:
(i) a solid support on which to bind a primary anti-PS antibody;
(ii) a solution containing the primary antibody;
(iii) at least one buffer solution to block unbound sites on the solid support and to wash the solid support;
(iv) a solution containing the labeled secondary antibody; and
(v) Positive and negative PS control cells.
